(19) United States
(12) Plant Patent Application Publication
(10) Pub. No.: US 2008/0184411 P1
(43) Pub. Date: Jul. 31, 2008

(54) (Plant) Buxus Sempervirens "Thomas Jefferson" a/k/a "Mr. Jefferson"

(76) Inventor: Thomas M. Patrick JR., Greenville, SC (US)

Correspondence Address: Thomas M Patrick Jr., PO Box 10351, Greenville, SC, 29603, US

(21) Appl. No.: 11/655,489

(22) Filed: Jan. 16, 2007

(51) Int. Cl. A01H 5/00

(52) U.S. Cl. PLT/226

(57) ABSTRACT

Subject Boxwood is a chance seedling from airborn pollination. Subject was discovered in a garden area at Charlottesville Va. about 1964. The Box' primary distinctive qualities is its rapid growth—as much as 5-7″ in both Spring and Fall; its large green-black (from color dictionary Elm Green) leaves; its dense foliage; and significance hardiness. Leaves are narrowly ovate with an obtuse apex and measure up to 15/16″ long and up to 7/16″ wide. The mother plant is now approximately 18′ tall and 13′ across. The Box reproduces readily from cuttings, from layering and it produces seed.

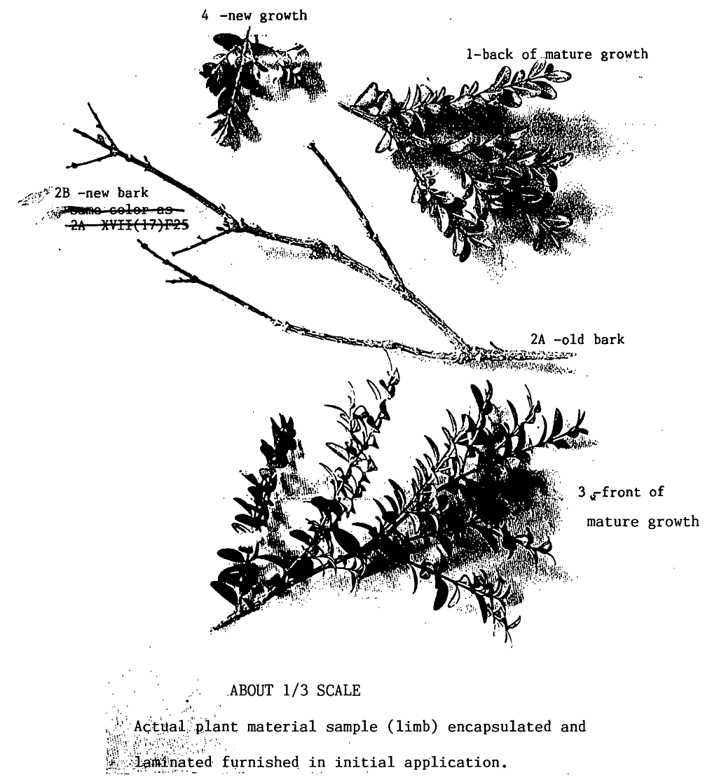

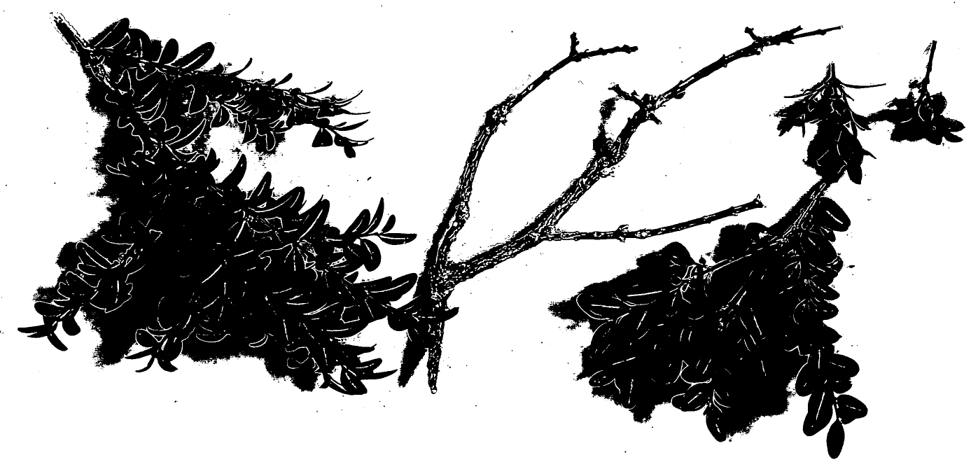

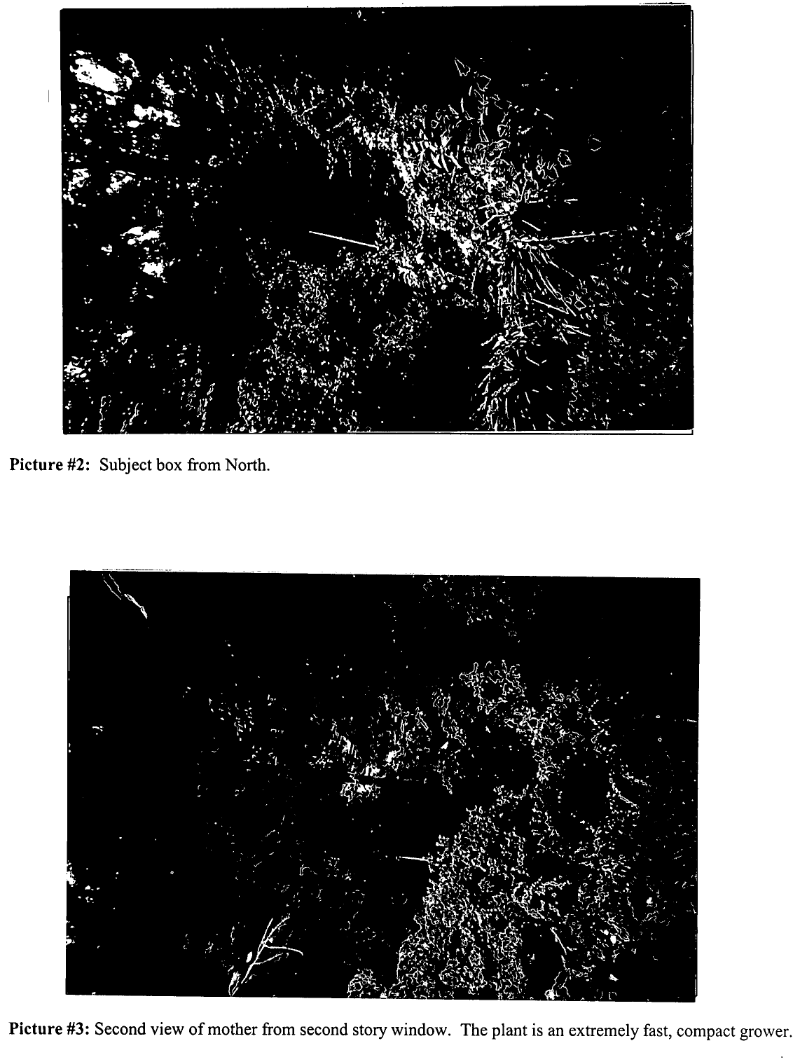

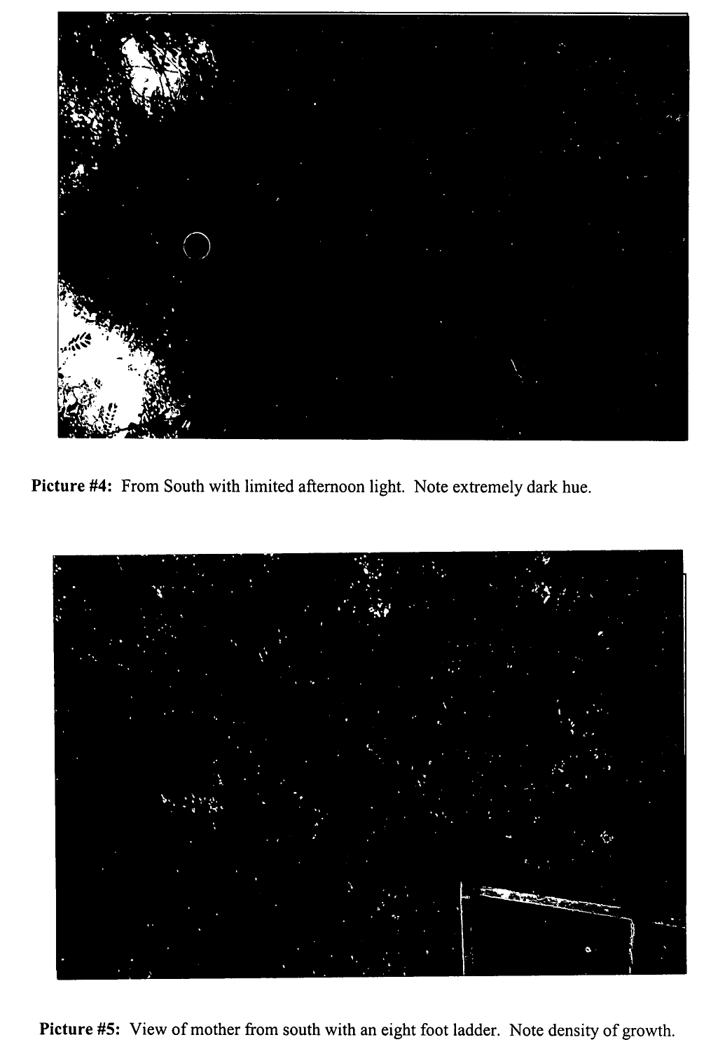

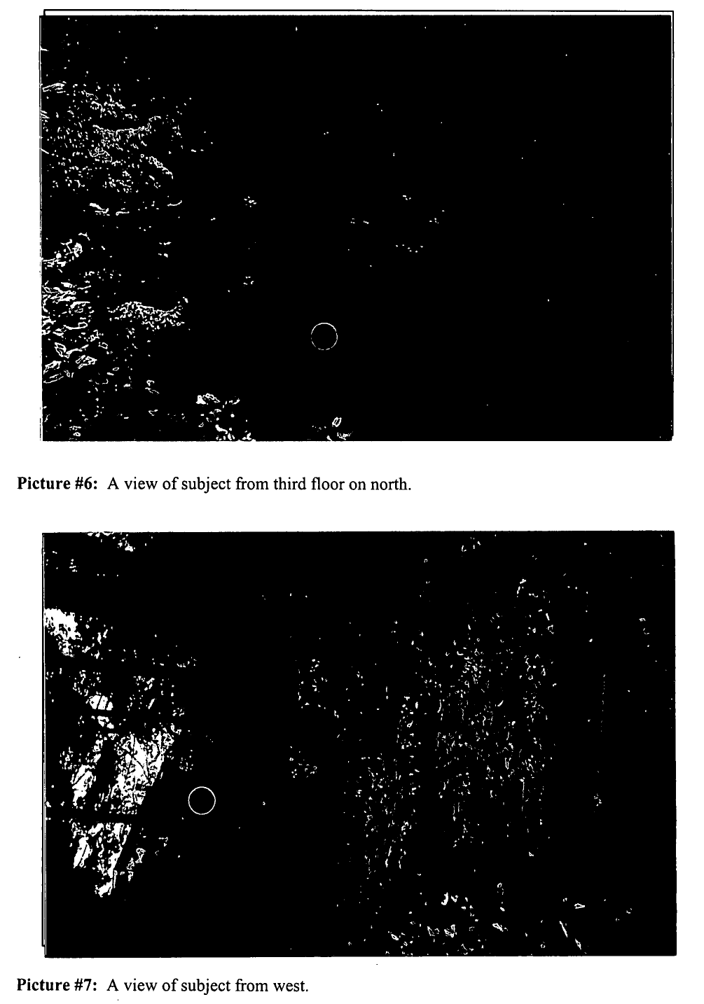

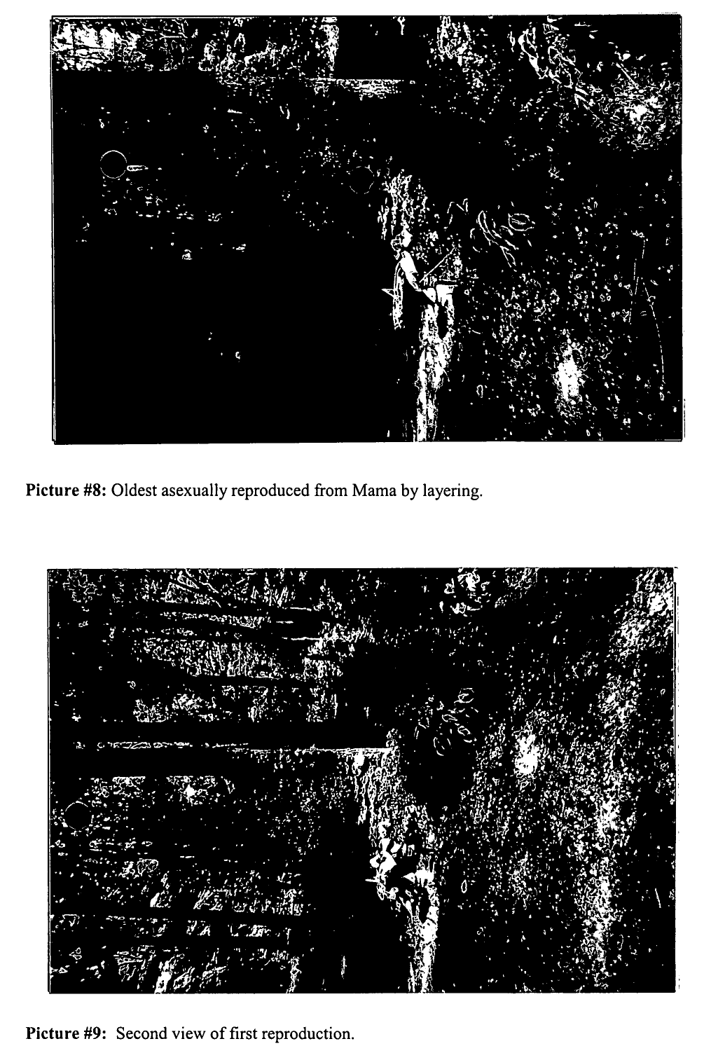

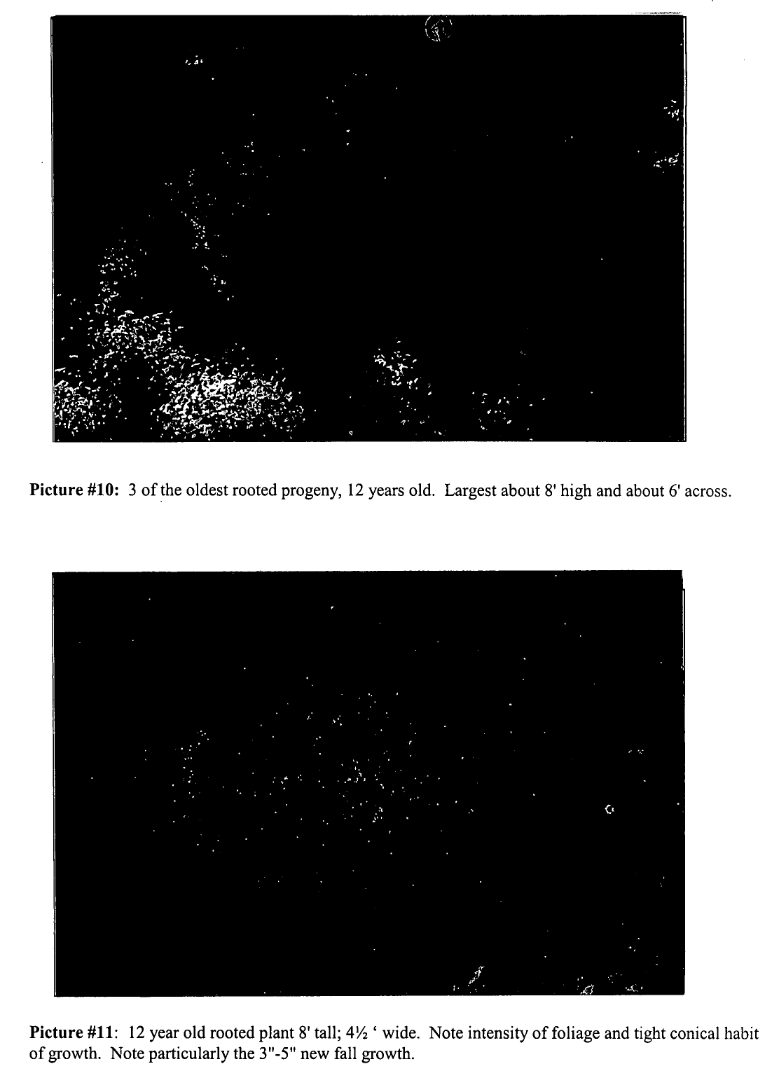

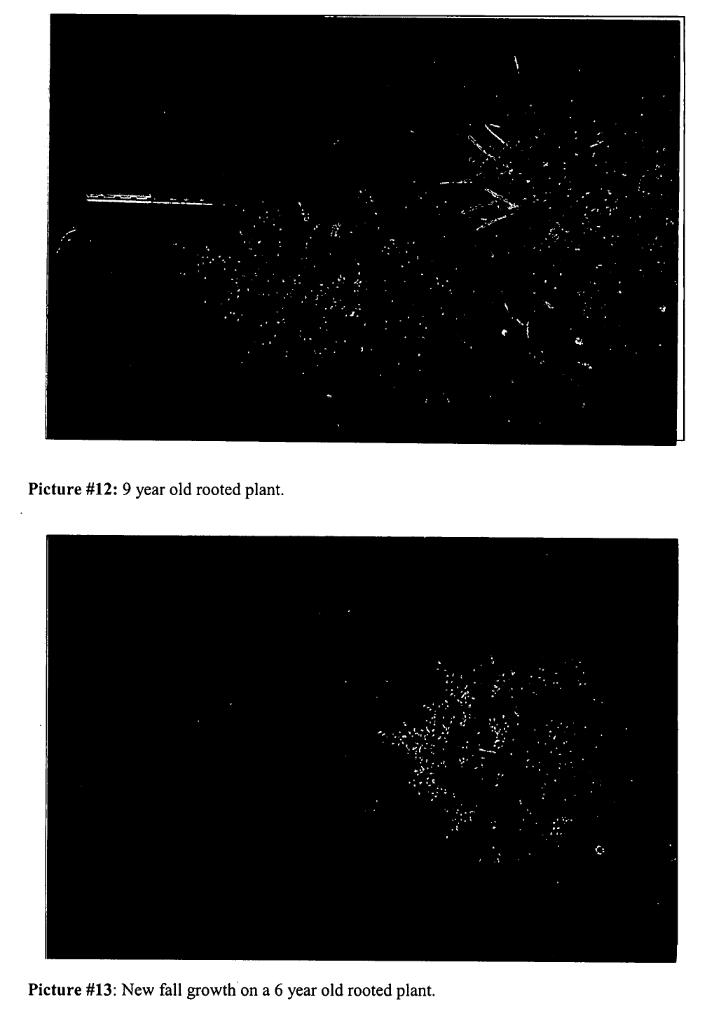

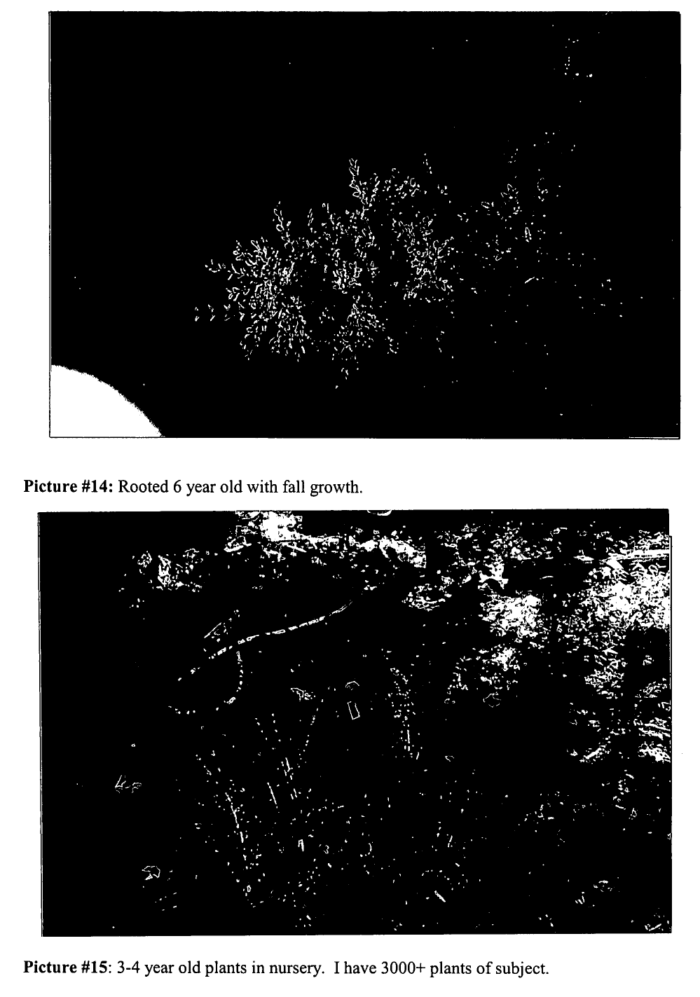

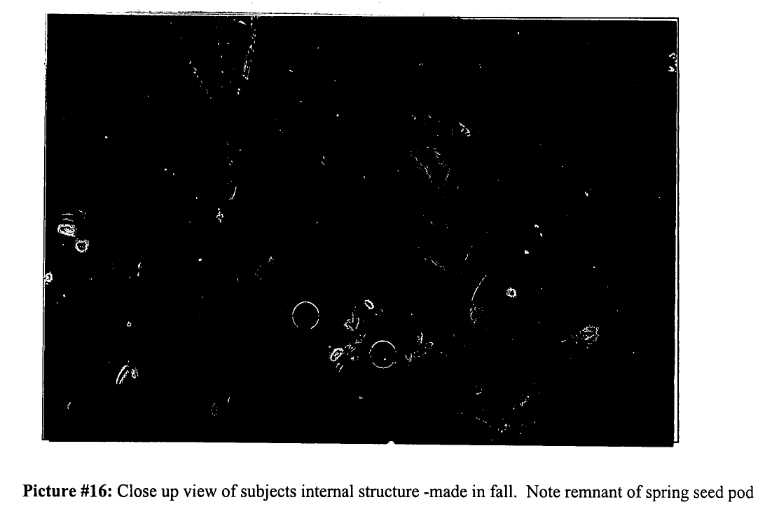

### ADDITIONAL BOTANICAL DESCRIPTION OF THE PLANT

A. Genus and Species:

Buxus Sempervirens “Thomas Jefferson” a/k/a “Mr. Jefferson”. A single trunked, upright shrub attaining a height of 15′ (4.5 m) and a width of 12″ (3.6 m) at 40 years of age. The leaves are arranged in a herringbone and collectively lie flat. They are relatively uniform being narrowly ovate with an obtuse apex. The leaves vary only slightly in size, being up to 15/16″ (16 m to 24 mm) long and up to 7/16″ (6 mm to 11 mm) wide. See F Botanical below. This was written by Boxwood registrar Lynn Batdorf in his listing report. Subject is now about 16′+ tall; 13′+ wide and specimen is laminated and color plate page 21.

B. Habit of Growth:

The growth habit is probably the most outstanding and different feature of subject from other boxwood. As you will see from photos 11-14, subject box puts on heavy new growth in the spring measuring from 3-7″ and also puts on a second new growth about August, early September which is usually somewhat shorter by an inch to an inch and a half. Remember, the subject plant is 44 years old and approximately 16′ tall so the box is average growing a little over 4″ a year. New growth on plants under five (5) years old is only 1½-2″ but at the sixth year speeds up to the extraordinary growth mentioned above. See pictures. Habit is dense; you will see from pictures that you normally cannot see thru the plant. The limbs initially rise at 45 degrees but become pendulent and stoop down with age. The samples of the boxwood material from subject is enclosed and it and the color dictionary entries show the intense and attractive black green color which we in South Carolina call Charleston Green (a green black). See color dictionary.

B. Cultivar Name:

Buxus Sempervirens “Thomas Jefferson” a/k/a “Mr. Jefferson”, the plant of Thomas M. Patrick Jr.

C. Vigor:

The habit of growth as referred to above and plant is extremely, extremely vigorous. It is not subject to any significant disease or susceptible to insects. It like most box, is free from deer munching. It is not subject in any significant way to any of the boxwood pests or diseases. If it is planted on a poor site, sometimes there is yellowing on the tips of some of the leaves but this can be readily handled generally by applications of iron and it does not persist if treated.

D. Productivity:

Plant can be readily reproduced in three and possibly four ways. By seed after pollination of the flower—the seeds are typical of box seed. The box is also readily reproduced asexually in two primary matters; rooting and layering. I have about 300 layered box and about 2700 rooted of subject. They transplant readily.

E. Precocity: N/A

F. Botanical:

Leaf structure on the branch: The leaf pair growths on a branch are not like the arrangement of leaves on American Boxwood. Those leaves are of successive pairs of leaves in a flat plane on the stem. On specimen, leaf structure and arrangement seems to be more like that that I have observed on English Boxwood which have successive pairs of leaves growing as much as ninety (90) degrees from previous set of leaves on the stem and to subsequent pairs on the stem.

The box appears to be a hardy and I believe it would be desirable in at least Zones 5-8. It has been hardy in Greenville S.C., Charlottesville Va., and Polk County NC. It does not bronze in winter. It's major winter problem is the fact that other plant material can fall on it and do damage but subject has survived without injury in major ice storms and blizzards.

One of the most interesting aspects of the plant is its new growth. In the first five (5) years, new growth approximates 1½-2½ inches in spring and up to 1½″ in the fall. At about six (6) years, new growth approximates from 5-7″ in the spring in light color sage gray growth which turns and hardens to the Charleston Green Black. See color chart page 21. These are layman terms—see attached photo on color charts. Surprisingly, and fairly uncommon in box, is the fact that subject plant and its progeny have late summer-fall new growth frequently but it does not occur every year—I think there is a weather variable. On the fall growth, subject puts on new growth (new foliage) of 3″+/− to 6 +/− and the growth has always harden before frost.

The surface of the mature leaves is glossy but not a high gloss. I would call it a semi-gloss. This categorization is much like in paint glosses.

Disease and conditions: The only condition that I am aware of is that periodically and isolated leaf tip yellowing which is based on the deficiency of iron or magnesium. This is easily remedied by appropriate chemical applications.

Pests: On a few weak plants and sites I had leaf miter damage and a few isolated mites; both easily treatable with systemics and/or horticultural oil.

Bark: The bark on stems and trunk is multilayered. The basic core of the stems is a light colored white wood. The next layer of the stem is a green—also the color of new growth as shown on color charts plate 17, page 21—Courage Green. The outer layer of the bark is somewhat quilted but has a predominant color as shown on color charts page 22—Light Brownish Olive. The bark on the outer bark sometimes flakes off exposing the green inner layer of the bark (very small area—usually about the size of a grain of uncooked rice. The configuration of the stems and trunk are typical of box with tip of new growth of second year growth being square, thus the name boxwood.

A single claim: We are only applying for one plant patent.

### DETAILED BOTANICAL DESCRIPTION

Reproduction: I have layered and rooted in both Greenville County, South Carolina and Polk County, North Carolina. A third and infrequent method of asexual reproduction comes from the fact that subject occasionally sprouts of new growth come up from the roots.

Leaf shape is not totally uniform but generally narrowly ovate with an obtuse apex. Leaves grow generally in pairs on each side of the stem. This type of growth is almost uniform in box. The leaves measure in length up to 15/16″ and in width up to 7/16″. The mother plant has three (3) stems the largest of which is 2″. It has three (3) upright stems but other plants occasionally have up to seven (7) or eight (8) upright stems, particularly if they have been layered. Most rooted plants have a single trunk. Limbs on the top of the plant are generally erect and parallel to the main stems; as limbs grow older they stoop down to 45° but the tips of the limbs generally point up in a slightly pendulum fashion.

Description of the Registrar Lynn Batdorf: A single trunked, upright shrub attaining a height of 15′ (4.5 m) and a width of 12″ (3.6 m) at 40 years of age. The leaves are arranged in a herringbone and collectively lie flat. They are relatively uniform being narrowly ovate with an obtuse apex. The leaves vary only slightly in size, being up to 15/16″ (16 m to 24 mm) long and up to 7/16″ (6 mm to 11 mm) wide.

G. Fertility:

The box blooms and bears seeds but I have been unable o get any at this point to germinate and I am not sure if any have germinated under that plants that are bearing seed. Box will also occasionally reproduce from roots (root sprouts) and it may be that some of the things that are root sprouts may be seedlings—it is very difficult to determine.

Subject box blooms in early spring. The blossoms are small and inconspicuous but very fragrant. The blossoms are greenish yellow MOMENCIOUS flowers.

The male (staminate) flowers have four sepals and four stamens that are much longer than the sepals. The female (distillate) flowers have six sepals and a three-celled ovary with three short styles. The fruit (capsule) is ovoid in shape. The capsule has three horns which can be separated into three pieces, each of which has two horns. Each piece holds two black shiny seeds for a total of six seeds per capsule.

The fruit capsules (see attached photo 16) appear fairly promptly and mature about the first week in July. They split open and there are six seed inside. On subject box, the seed pods appear random, adjacent to stem at base of leaf. They are about ½ the size of an average rose hip. Leaf buds generally appear also adjacent to the stem at the base of a leaf. The nascent flower (see photo) has appearance very much like a soccer ball or like a crown. Globular flower buds are about ⅓ the size of an eraser on the end of pencil.

New growth commences about the 15^{th} of April in spring and remember subject box also often—apparently very rare to box generally—puts out substantial new growth in fall. Flower blooms in early spring—depending on location—between April Fool's Day and about the 10^{th} of May. These observations are shown more fully in captions to pictures sent.

Six black seeds about the size of a lead on a sharpened wooden pencil or a peppercorn.

H. Other Characteristics:

I am attaching for other characteristics the narrative above that goes into great detail all aspects of the plant. The plant flowers about April Fool's Day up to early May and seed matures in late June or early July. The plant prefers well drained low soil; it seems to be somewhat drought resistant but in the last couple of summers when we had extended drought, I did some watering on the rootings. The box is cold hardy at least in South Carolina, North Caroline and Virginia and I believe it could probably go up to as far north as Philadelphia, Pa. and Ohio and whatever that zone is. I doubt it would do well in the south below the Florida-Georgia line but these are fairly typical ranges for hardy boxwood. The box will also do well in moist situations. Some of the box shown on the pictures are planted over an underground creek and they do okay. The only weather damage that they are perceptible to are to limbs falling on them from trees up above. I have had no damage from snow or even extended ice. The limbs are very supple.

1. Subject Boxwood is a chance seedling from airborne pollination. Subject was discovered in garden area in Charlottesville, Va. about 1984 and was, at the time, a seedling of approximately 3″ tall. The Box' primary distinctive qualities are: 1) rapid but dense growth—as much as 5″-7″ in both spring and fall; 2) large green black (from color dictionary Elm Green) leaves, up to 15/16″ long and 7/16″ wide; 3) dense foliage; and 4) extreme hardiness to cold, drought, insect and disease conditions. The tree is particularly appealing to nursery growers, since as is evident from pictures, it has a natural “Christmas tree shape” that occurs naturally without the necessity of pruning. None of the pictures in the application are of subject plants that have ever been trimmed.
